(19) United States
(12) Patent Application Publication
(10) Pub. No.: US 2026/0207854 A1
(43) Pub. Date: Jul. 23, 2026

(54) Protective Cover for Dental Syringe

(71) Applicant: Emmad Sheikh, Murphy, TX (US)

(72) Inventor: Emmad Sheikh, Murphy, TX (US)

(21) Appl. No.: 19/031,778

(22) Filed: Jan. 18, 2025

(51) Int. Cl. A61M 5/32

(52) U.S. Cl. CPC A61M 5/3204; A61M 5/3293; A61M 2005/3247; A61M 2210/0637

(57) ABSTRACT

A protective cover configured to be removably coupled to a dental syringe having a needle hub is disclosed. The protective cover includes a coupler portion configured to be removably coupled to the dental syringe. The coupler portion defines a first end, a second end, and a cavity configured to receive the needle hub. The protective cover includes a tube assembly extending away from the second end of the coupler portion such that the tube assembly at least partially cover a needle of the dental syringe. The tube assembly includes a first tube extending downwardly from the second end of the coupler portion, a second tube extending inclinedly from the first tube, and a third tube slidably coupled to the first tube and configured to at least partially cover the needle. The third tube is configured to move in an extended position and a retracted position relative to the first tube.

### TECHNICAL FIELD

This disclosure relates generally to a dental syringe, and more particularly to a protective cover configured to be coupled to a dental syringe.

### BACKGROUND

Dental procedures often induce anxiety in children, particularly due to fear of dental syringes used for administering local anesthesia. This fear is largely due to visibility of needle in the dental syringes, which children associate with pain. As a result, the children may become anxious, uncooperative, or even physically resist treatment when the syringe is introduced. Such reactions may complicate a dentist's task and increase the risk of accidental injury, both to the child and the dentist, due to sudden movements of the children during the procedure. While various methods have been employed to reduce dental anxiety in children, such as distraction techniques, sedation, and smaller needle sizes, there remains a significant challenge in preventing the initial fear triggered by the sight of the needle.

Therefore, there is need for a dental syringe which not only mitigates fear and anxiety in children but also protect them from any injury, thereby creating a more comfortable and less stressful experience for children.

### SUMMARY OF INVENTION

In an embodiment, a protective cover configured to be removably coupled to a dental syringe having a needle hub is disclosed. The protective cover includes a coupler portion configured to be removably coupled to the dental syringe. The coupler portion defines a first end, a second end, and a cavity configured to receive the needle hub of the dental syringe. The protective cover further includes a tube assembly extending away from the second end of the coupler portion such that the tube assembly at least partially cover a needle of the dental syringe. The tube assembly includes a first tube extending downwardly from the second end of the coupler portion, a second tube extending inclinedly from the first tube, and a third tube slidably coupled to the first tube and configured to at least partially cover the needle of the dental syringe. The third tube is configured to move in an extended position and a retracted position relative to the first tube. In the extended position, the third tube completely covers the needle of the dental syringe. In the retracted position, the third tube exposes the needle of the dental syringe to enable anesthesia injection to desired area.

In an embodiment, the tube assembly further includes a biasing member configured to bias the third tube in the extended position.

In an embodiment, the third tube includes a handle configured to be operated by a user to move the third tube in the retracted position from the extended position to facilitate the slidable movement of the third tube relative to the first tube.

In an embodiment, the first tube includes an engagement structure and the third tube includes a retention structure configured to be engaged with the engagement structure of the first tube to facilitate the slidable coupling of the third tube with the first tube.

In an embodiment, the engagement structure is a slot and the retention structure is a protrusion configured to be engaged with the slot.

In an embodiment, the second tube is fluidly connected to a water supply to enable a water flow inside the tube assembly such that the water flows inside the tube assembly via the second tube and exits the tube assembly via the third tube.

In an embodiment, the water flowing inside tube assembly and exiting the tube assembly via the third tube is used to perform a cleaning operation of desired area.

In an embodiment, a dental syringe assembly is disclosed. The dental syringe assembly includes a dental syringe including a needle hub having a needle and a protective cover removably coupled to the needle hub of the dental syringe. The protective cover includes a coupler portion configured to be removably coupled to the dental syringe. The coupler portion defines a first end, a second end, and a cavity configured to receive the needle hub of the dental syringe. The protective cover further includes a tube assembly extending away from the second end of the coupler portion such that the tube assembly at least partially cover a needle of the dental syringe. The tube assembly includes a first tube extending downwardly from the second end of the coupler portion, a second tube extending inclinedly from the first tube, and a third tube slidably coupled to the first tube and configured to at least partially cover the needle of the dental syringe. The third tube is configured to move in an extended position and a retracted position relative to the first tube. In the extended position, the third tube completely covers the needle of the dental syringe. In the retracted position, the third tube exposes the needle of the dental syringe to enable anesthesia injection to desired area

In an embodiment, the tube assembly further includes a biasing member configured to bias the third tube in the extended position.

In an embodiment, the third tube includes a handle configured to be operated by a user to move the third tube in the retracted position from the extended position to facilitate the slidable movement of the third tube relative to the first tube.

In an embodiment, the first tube includes an engagement structure and the third tube includes a retention structure configured to be engaged with the engagement structure of the first tube to facilitate the slidable coupling of the third tube with the first tube.

In an embodiment, the engagement structure is a slot and the retention structure is a protrusion configured to be engaged with the slot.

In an embodiment, the second tube is fluidly connected to a water supply to enable a water flow inside the tube assembly such that the water flows inside the tube assembly via the second tube and exits the tube assembly via the third tube.

In an embodiment, the water flowing inside tube assembly and exiting the tube assembly via the third tube is used to perform a cleaning operation of desired area.

It is to be understood that both the foregoing general description and the following detailed description are exemplary and explanatory only and are not restrictive of the invention, as claimed.

### BRIEF DESCRIPTION OF THE DRAWINGS

The present application can be best understood by reference to the following description taken in conjunction with the accompanying drawing figures, in which like parts may be referred to by like numerals.

FIG. 1 illustrates a perspective view of a dental syringe assembly including a dental syringe and a protective cover, in accordance with an embodiment of the present disclosure.

FIG. 2 illustrates a perspective view of the protective cover of FIG. 1, in accordance with an embodiment of the present disclosure.

FIG. 3 illustrates a perspective view of the protective cover of FIG. 1 in an extended position, in accordance with an embodiment of the present disclosure.

FIG. 4 illustrates a perspective view of the protective cover of FIG. 1 in a retracted position, in accordance with an embodiment of the present disclosure.

### DETAILED DESCRIPTION OF THE DRAWINGS

The following description is presented to enable a person of ordinary skill in the art to make and use the invention and is provided in the context of particular applications and their requirements. Various modifications to the embodiments will be readily apparent to those skilled in the art, and the generic principles defined herein may be applied to other embodiments and applications without departing from the spirit and scope of the invention. Moreover, in the following description, numerous details are set forth for the purpose of explanation. However, one of ordinary skill in the art will realize that the invention might be practiced without the use of these specific details. In other instances, well-known structures and devices are shown in block diagram form in order not to obscure the description of the invention with unnecessary detail. Thus, the invention is not intended to be limited to the embodiments shown, but is to be accorded the widest scope consistent with the principles and features disclosed herein.

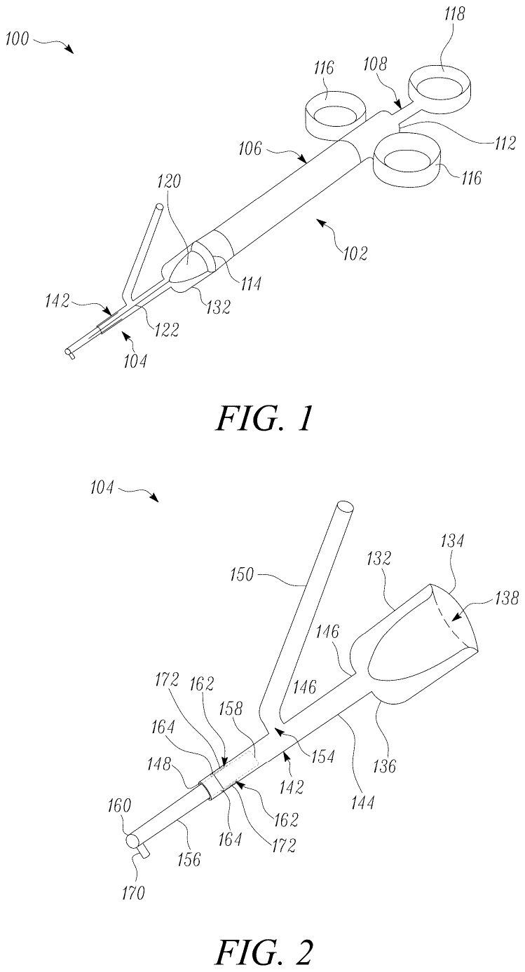

Referring to FIG. 1, a perspective view of a dental syringe assembly 100 is illustrated, in accordance with an embodiment of the present disclosure. The dental syringe assembly 100 includes a dental syringe 102 and a protective cover 104 removably coupled to the dental syringe 102. The dental syringe 102 is primarily used for administering local anesthesia to ensure patient comfort during various dental procedures. However, the dental syringe 102 may also be used for dispensing dental materials, like bonding agent, impression material, and/or cement.

As shown in FIG. 1, the dental syringe 102 includes a hollow elongated barrel 106 and a plunger 108 configured to be housed and move within the barrel 106. It should be noted that the barrel 106 is designed to store an anesthesia solution. Further, the barrel 106 includes a first barrel end 112, a second barrel end 114 disposed opposite to the first barrel end 112, and a holder portion 116 disposed proximal to the second barrel end 114. As shown, the holder portion 116 is shaped as a pair of finger grips configured to facilitate holding of the dental syringe 102 in a hand of a user. Further, the first barrel end 112 may be open to receive the plunger 108. The plunger 108 is slidably disposed within the barrel 106 such that the plunger 108 is movable therein between a first position and a second position.

The plunger 108 includes a pusher portion 118 extending out from the first barrel end 112. As shown, the pusher portion 118 is shaped as a thumb ring configured to facilitate pushing of the plunger 108 between the first position and the second position. In the first position, the pusher portion 118 may be positioned farther from the first barrel end 112. In the second position, the pusher portion 118 may be positioned near the first barrel end 112. In other words, during the movement of the plunger 108 from the first position to the second position, the pusher portion 118 moves towards the first barrel end 112.

It may be appreciated that during movement of the plunger 108 from the first position to the second position the anesthesia solution may be dispensed from the dental syringe 102 and may be injected in a desired area. The dental syringe 102 further includes a needle hub 120 removably coupled to the second barrel end 114. The needle hub 120 includes a needle 122 which may be used for injecting the anesthesia solution to the desired area. The protective cover 104 is configured to be removably coupled to the needle hub 120 of the dental syringe 102.

Referring to FIG. 2, a perspective view of the protective cover 104 is illustrated, in accordance with an embodiment of the present disclosure. The protective cover 104 includes a coupler portion 132 configured to be removably coupled to the dental syringe 102. Specifically, the coupler portion 132 is removably coupled to the needle hub 120 of the dental syringe 102. In some embodiments, the coupler portion 132 is threadably coupled with the needle hub 120 of the dental syringe 102. The coupler portion 132 defines a first end 134, a second end 136 disposed opposite to the first end 134, and a cavity 138 formed therein. The first end 134 of the coupler portion 132 is removably coupled to the needle hub 120 of the dental syringe 102. Moreover, the cavity 138 is shaped such that the needle hub 120 is received within the cavity 138.

Further, the protective cover 104 includes a tube assembly 142 extending away from the second end 136 of the coupler portion 132 such that the tube assembly 142 at least partially cover the needle 122 of the dental syringe 102. The tube assembly 142 includes a first tube 144 extending downwardly from the second end 136 of the coupler portion 132. The first tube 144 includes a first end 146 coupled to the second end 136 of the coupler portion 132 and a second end 148 disposed opposite to the first end 146. The tube assembly 142 further includes a second tube 150 extending inclinedly from the first tube 144. In some embodiments, the second tube 150 is connected proximal to the second end 148 of the first tube 144.

The second tube 150 is fluidly connected to a water supply to enable a water flow inside the tube assembly 142 via a passage 154. The tube assembly 142 further includes a third tube 156 slidably coupled to the first tube 144 such that the water entering inside the tube assembly 142 via the second tube 150 exits via the third tube 156. The third tube 156 also facilitates a selective entry of the water inside the tube assembly 142 by opening and closing the passage 154, which will be described later in the disclosure.

The third tube 156 includes a first end 158 slidably coupled to the second end 148 of the first tube 144 and a second end 150 disposed opposite to the first end 158 and extending outwardly of the first tube 144. The second end 160 of the third tube 156 is a free end. To facilitate the slidable coupling of the third tube 156 with the first tube 144, the first tube 144 includes an engagement structure 162 and the third tube 156 includes a retention structure 164 configured to be engaged with the engagement structure 162 of the first tube 144.

In some embodiments, the engagement structure 162 is a slot and the retention structure 164 is a protrusion configured to be engaged with the slot. Without restricting the scope of the disclosure, it would be appreciated that instead of slot-protrusion mechanism, any suitable mechanism for enabling the slidable movement between the first tube 144 and the third tube 156 may be used. It may be appreciated that the engagement structure 162 may be disposed at an inner surface of the first tube 144 and the retention structure 164 may be disposed at an outer surface of the third tube 156. Further, the third tube 156 is configured to be moved in an extended position P1 and a retracted position P2 relative to the first tube 144.

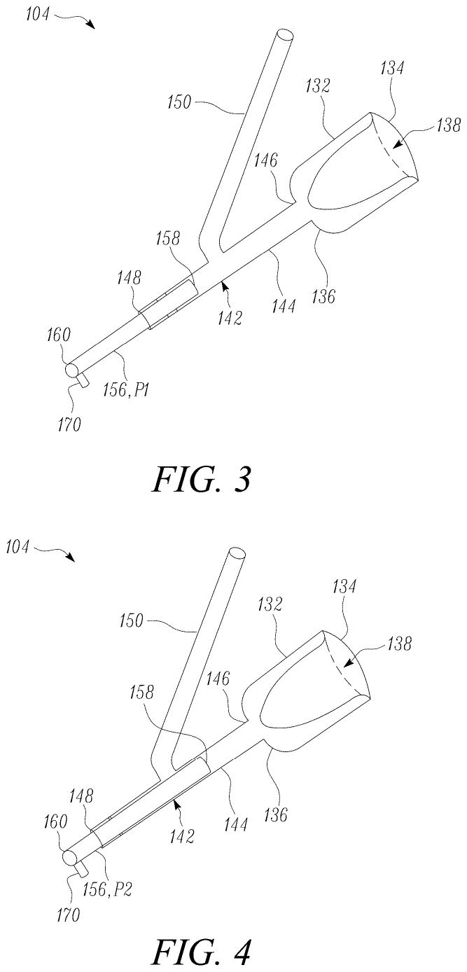

In FIG. 3, the protective cover 104 is shown in the extended position P1 and in FIG. 4, the protective cover 104 is shown in the retracted position P2. As shown in FIG. 3, in the extended position P1, the second end 160 of the third tube 156 is positioned farther from the first tube 144 (i.e., second end 148), thereby completely covering the needle 122 of the dental syringe 102. Moreover, in the extended position P1, the first end 158 of the third tube 156 is positioned such that the passage 154 is open. Accordingly, the water flows inside the tube assembly 142 in the extended position P1.

Further, in the retracted position P2 (as shown in FIG. 4), the second end 160 of the third tube 156 is positioned near the first tube 144 (i.e., second end 148) exposing the needle 122 of the dental syringe 102 to enable the anesthesia injection to the desired area. Moreover, in the retracted position P2 the first end 158 of the third tube 156 is disposed inside the first tube 144 such that it closes the passage 154. Accordingly, the water flow inside the tube assembly 142 is prevented in the retracted position, thereby the anesthesia injection process may be carried out smoothly.

Inclusion of the second tube 150 in the protective cover 104 enables dual functioning of the dental syringe 102. The dental syringe 102 may also be used as water jet flosser to perform cleaning operation. Moreover, the slidable movement of the third tube 156 inside the first tube 144 enables the dental syringe 102 to perform selective operation at a time. In the extended position P1, the dental syringe 102 may operate as the water jet flosser and in the retracted position P2, as the water flow is prevented, the dental syringe 102 may be used for anesthesia injection without a need to switch to a different instrument. Further, the third tube 156 covers the needle 122 of the dental syringe 102, thereby reduces anxiety and fear that children typically experience during dental procedure.

Additionally, or optionally, the second tube 150 may include a valve (not shown) to regulate the water flow inside the tube assembly 142. Further, to enable the slidable movement of the third tube 156 within the first tube 144 between the extended position P1 and the retracted position P2, the third tube 156 includes a handle 170 configured to be operated by a user. The user may hold and push the handle 170 towards the first tube 144 to move the third tube 156 in the retracted position from the extended position relative to the first tube.

Moreover, the protective cover 104 further includes a biasing member 172 configured to bias the third tube 156 in the extended position P1. Normally, the third tube 156 is disposed in the extended position P1. In order to put the third tube 156 into the retracted position P2, the user has to push the handle 170 against the biasing member 172. Further, upon releasing of the handle 170 by the user, the third tube 156 is come into the extended position P1 due to action of the biasing member. In the illustrated embodiment of FIG. 2, the biasing member 172 is a spring.

It will be appreciated that, for clarity purposes, the above description has described embodiments of the invention with reference to different functional units and processors. However, it will be apparent that any suitable distribution of functionality between different functional units, processors or domains may be used without detracting from the invention. For example, functionality illustrated to be performed by separate processors or controllers may be performed by the same processor or controller. Hence, references to specific functional units are only to be seen as references to suitable means for providing the described functionality, rather than indicative of a strict logical or physical structure or organization.

Although the present invention has been described in connection with some embodiments, it is not intended to be limited to the specific form set forth herein. Rather, the scope of the present invention is limited only by the claims. Additionally, although a feature may appear to be described in connection with particular embodiments, one skilled in the art would recognize that various features of the described embodiments may be combined in accordance with the invention.

Furthermore, although individually listed, a plurality of means, elements or process steps may be implemented by, for example, a single unit or processor. Additionally, although individual features may be included in different claims, these may possibly be advantageously combined, and the inclusion in different claims does not imply that a combination of features is not feasible and/or advantageous. Also, the inclusion of a feature in one category of claims does not imply a limitation to this category, but rather the feature may be equally applicable to other claim categories, as appropriate.

What is claimed is:

1. A protective cover configured to be removably coupled to a dental syringe having a needle hub, the protective cover comprising:
a coupler portion configured to be removably coupled to the dental syringe, the coupler portion defining a first end, a second end, and a cavity configured to receive the needle hub of the dental syringe; and
a tube assembly extending away from the second end of the coupler portion such that the tube assembly at least partially cover a needle of the dental syringe, the tube assembly comprising:
a first tube extending downwardly from the second end of the coupler portion;
a second tube extending inclinedly from the first tube; and
a third tube slidably coupled to the first tube and configured to at least partially cover the needle of the dental syringe, the third tube is configured to move in an extended position and a retracted position relative to the first tube, wherein
in the extended position, the third tube completely covers the needle of the dental syringe, and
in the retracted position, the third tube exposes the needle of the dental syringe to enable anaesthesia injection to desired area.

2. The protective cover of claim 1, wherein the tube assembly further comprises a biasing member configured to bias the third tube in the extended position.

3. The protective cover of claim 1, wherein the third tube comprising a handle configured to be operated by a user to move the third tube in the retracted position from the extended position to facilitate the slidable movement of the third tube relative to the first tube.

4. The protective cover of claim 1, wherein the first tube comprises an engagement structure and the third tube comprises a retention structure configured to be engaged with the engagement structure of the first tube to facilitate the slidable coupling of the third tube with the first tube.

5. The protective cover of claim 4, wherein the engagement structure is a slot and the retention structure is a protrusion configured to be engaged with the slot.

6. The protective cover of claim 1, wherein the second tube is fluidly connected to a water supply to enable a water flow inside the tube assembly such that the water flows inside the tube assembly via the second tube and exits the tube assembly via the third tube.

7. The protective cover of claim 6, wherein the water flowing inside tube assembly and exiting the tube assembly via the third tube is used to perform a cleaning operation of desired area.

8. A dental syringe assembly, comprising:
a dental syringe comprising a needle hub having a needle; and
a protective cover removably coupled to the needle hub of the dental syringe, the protective cover comprising:
a coupler portion configured to be removably coupled to the dental syringe, the coupler portion defining a first end, a second end, and a cavity configured to receive the needle hub of the dental syringe; and
a tube assembly extending away from the second end of the coupler portion such that the tube assembly at least partially cover the needle of the dental syringe, the tube assembly comprising:
a first tube extending downwardly from the second end of the coupler portion;
a second tube extending inclinedly from the first tube; and
a third tube slidably coupled to the first tube and configured to at least partially cover the needle of the dental syringe, the third tube is configured to move in an extended position and a retracted position relative to the first tube, wherein
in the extended position, the third tube completely covers the needle of the dental syringe, and
in the retracted position, the third tube exposes the needle of the dental syringe to enable anesthesia injection to desired area.

9. The dental syringe assembly of claim 8, wherein the tube assembly further comprises a biasing member configured to bias the third tube in the extended position.

10. The dental syringe assembly of claim 8, wherein third tube comprising a handle configured to be operated by a user to move the third tube in the retracted position from the extended position to facilitate the slidable movement of the third tube relative to the first tube.

11. The dental syringe assembly of claim 8, wherein the first tube comprises an engagement structure and the third tube comprises a retention structure configured to be engaged with the engagement structure of the first tube to facilitate the slidable coupling of the third tube with the first tube.

12. The dental syringe assembly of claim 11, wherein the engagement structure is a slot and the retention structure is a protrusion configured to be engaged with the slot.

13. The dental syringe assembly of claim 8, wherein the second tube is fluidly connected to a water supply to enable a water flow inside the tube assembly such that the water flows inside the tube assembly via the second tube and exits the tube assembly via the third tube.

14. The dental syringe assembly of claim 13, wherein the water flowing inside tube assembly and exiting the tube assembly via the third tube is used to perform a cleaning operation of desired area.
